(19) United States
(12) Patent Application Publication
(10) Pub. No.: US 2023/0157666 A1
(43) Pub. Date: May 25, 2023

(54) SYSTEM FOR ROBOT-ASSISTED ULTRASOUND SCANNING

(71) Applicant: Life Science Robotics ApS, Aalborg (DK)

(72) Inventors: Rune Kristensen, Aalborg (DK); Jon David Ragnarsson, Aalborg (DK); Birgitte Størup, Aalborg (DK)

(73) Assignee: Life Science Robotics ApS, Aalborg (DK)

(21) Appl. No.: 17/971,426

(22) Filed: Oct. 21, 2022

(30) Foreign Application Priority Data
Nov. 24, 2021 (DK) PA202170583

(51) Int. Cl. A61B 8/00; A61B 8/08; A61B 34/30

(52) U.S. Cl. CPC A61B 8/4416; A61B 8/4483; A61B 8/461; A61B 8/483; A61B 34/30; A61B 2090/064

(57) ABSTRACT

A system for robot-assisted ultrasound scanning comprises a multi axis robot with an end effector, a transducer holding element for connecting an ultrasound transducer to the end effector, a user input arrangement having at least three separate proportional inputs representing desired ultrasound transducer displacement along X, Y and Z axes of an orthogonal coordinate system defining the ultrasound transducer motion, and a controller which is connected to the user input arrangement and which controls the end effector based on input. The controller acquires a 3D model of a surface to be scanned and is arranged to continuously update the orthogonal coordinate system defining the ultrasound transducer motion as the ultrasound transducer moves, the X, Y and Z axes are arranged as described. In this way, the operator can easily move the transducer along the surface of the area to be scanned.

### FIELD

The field relates to a system for robot-assisted ultrasound scanning.

### DESCRIPTION OF RELATED ART

Ultrasound scanning is used in many different applications. One common and well-known application is to scan pregnant women to observe and/or diagnose the foetus in the womb. However, many other applications are known in the art.

When doing ultrasound scans, for example scans of a pregnant woman, it is typical that an operator will manually guide a handheld ultrasound transducer over a patient's skin. The operator will move the transducer around, change the orientation of the transducer with respect to the patient, as well as press the transducer firmly against the skin of the patient. Depending on the images produced by the ultrasound transducer, the operator will need to move the transducer to another location, change the orientation of the transducer and/or press or release pressure on the skin of the patient.

During a typical ultrasound scanning procedure, the operator will usually have to apply quite a bit of force to the transducer via his or her arm and hand. Furthermore, in many cases, the operator will also be sitting or standing in an awkward position. This results in unhealthy stresses on the body of the operator. It is therefore often the case, that operators of ultrasound transducers will experience physical problems which can cause short term sick leave from work, as well as long term wear and/or damage to their bodies. Shoulder, elbow, wrist and/or back strains are very common.

This is a known issue in the medical industry, but has yet to be solved effectively. A number of different solutions have been proposed where it is suggested to use robots to position the ultrasound transducer on the skin of the patient and then via a form of control device, for example a joystick, allow an operator to guide the robot to move the ultrasound transducer over the area to be scanned. Some examples of such systems are disclosed in WO2019229099, US2008021317, U.S. Pat. No. 6,425,865 and US2017252002. Many of the prior art solutions use force feedback on the input device to allow the operator to feel the amount of force being applied to the patient. In other solutions, a computer is provided to completely control the motion according to predefined paths or traces stored in the system. In certain cases, these predefined paths can be optimized and adapted based on physical details of the patient being scanned. In other cases, the operator is provided with a joystick which can operate the end effector of the robot in six dimensions (x,y,z+rotations about each axis). In many cases, the robots are provided with safety mechanisms which measure the force being applied by the robot to the patient and which stop the motion of the robot if predetermined limits are passed. Such robot assistant ultrasound systems are often suggested for telemetry operations where the ultrasound operator is located in a location where he/she does not have visual connection with the area to be scanned.

However, the prior art systems are all rather difficult to use for many normal applications. There therefore remains room for improvement.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A basic arrangement comprises a system for robot-assisted ultrasound scanning comprises a multi axis robot with an end effector, said multi axis robot and end effector being arranged such that said end effector can be moved in at least six degrees of freedom, a transducer holding element suitable for connecting an ultrasound transducer to the end effector of the multi axis robot in a known position with respect to the end effector, a user input arrangement having at least three separate proportional inputs, said at least three separate proportional inputs representing desired ultrasound transducer displacement along X, Y and Z axes of an orthogonal coordinate system defining the ultrasound transducer motion, and a controller which is connected to the user input arrangement and controls the end effector of the multi axis robot, based on the input from the user input arrangement.

It is therefore a first aspect to provide a system like the basic arrangement, which is easier to operate for an operator.

It is a second aspect to provide a system like the basic arrangement, which allows for a much-reduced physical load on the operator.

These aspects are provided at least in part via a system like the basic arrangement being further characterized according to the features of the characterized portion of claim 1.

In this way, a system is provided where it is very easy for the operator to control the motion of the transducer on the surface to be scanned, without having to manually control all the degrees of freedom of the robot.

It should be noted that by user input arrangement is meant an arrangement of one or more input devices. This could either be multiple separate devices working together or a single multi degree of freedom device which allows a user to provide input to the controller. In one embodiment, the user input arrangement is a single mechanical input device having six degrees of freedom, for example a device marketed under the brand “3Dconnexion” and with the product name of “SpaceMouse”. Other suitable devices with six degrees of freedom are known in the art. For the sake of this specification, the term proportional input is meant an input which a user can manipulate and which can provide an output with a variable output which is proportional to the motion of an input device. A typical example of a proportional input device is a joystick which is operatable in two separate directions to provide proportional inputs in 2 separate directions. In one embodiment, the transducer holding element is suitable for detachably connecting an ultrasound transducer to the end effector.

Within the scope of the invention, the user input arrangement could also include display devices and/or additional controllers which translate desired motion from one coordinate system to another. The user input arrangement could also comprise additional means to make control of the transducer easier for the operator.

In one embodiment, the user input arrangement comprises at least three separate proportional inputs, said at least three separate proportional inputs representing the desired ultrasound transducer displacement along the X, Y and Z axes of the transducer orthogonal coordinate system defining the ultrasound transducer motion. In this way, the user can easily control the motion of the transducer with respect to the 3D model of the surface.

In one embodiment, the X-axis of the orthogonal coordinate system defining the ultrasound transducer motion is arranged on a plane which is parallel to an X-Z plane of a global fixed orthogonal coordinate system. In one embodiment, the Y-axis of the orthogonal coordinate system defining the ultrasound transducer motion is arranged on a plane which is parallel to a Y-Z plane of a global fixed orthogonal coordinate system. In one embodiment, the X-Z plane of the global fixed orthogonal coordinate system is arranged perpendicular to the sagittal plane of the patient. In this way, motion along the X axis will always be on a plane which is perpendicular to the sagittal plane. Likewise, in one embodiment, the Y-Z plane is arranged parallel to the sagittal plane of the patient. In this way motions along the Y axis will always be on a plane which is parallel to the sagittal plane.

In one embodiment, the system comprises a 3D scanning arrangement which is arranged to scan a surface of the area to be scanned and to generate a 3D model of the surface of the area to be scanned. In one embodiment, the surface scanning device comprises a 3D laser scanner. In another embodiment, the surface scanning device comprises at least two cameras, placed at a distance from each other. In one embodiment, the surface scanning device is connected to the end effector of the multi axis robot and scans the area around the ultrasound transducer.

In one embodiment, the user input arrangement has at least one, at least two or at least three additional proportional input(s) representing rotation of the ultrasound transducers about at least one, two or three separate axes respectively and in that the controller is arranged to apply those inputs to rotate the ultrasound transducer about said X, Y and Z axes respectively.

In one embodiment, the system comprises a display, said display displaying a virtual representation of the ultrasound transducer on a representation of the area to be scanned.

In one embodiment, the system comprises a camera suitable for capturing an image of the area to be scanned and in that the representation of the area to be scanned is the image of the area to be scanned. In one embodiment, the 3D model is used as the representation of the area to be scanned. In one embodiment, the 3D model is combined with the image captured by the camera to create a combined 3D model and image.

In one embodiment, the system further comprises a force sensor which measures the force applied to the ultrasound transducer or a force estimator which estimates the force applied to the ultrasound transducer. In one embodiment, the multi axis robot is provided with multiple force sensors, one provided at each actuator and/or joint, the controller being arranged to determine the force applied to the end effector via the knowledge of the geometry of the robot and the forces measured at each actuator and/or joint. In one embodiment, the system comprises a separate force transducer arranged between the end effector of the robot and the ultrasound transducer.

In one embodiment, the system is arranged to stop motion of the robot when the measured or estimated force exceeds predefined limits and/or in that the system is arranged to reflect the measured or estimated force back to the user via a force feedback mechanism in the user input arrangement. In one embodiment, at least one proportional input of the user input arrangement is a mechanical input device which is displaceable along or about an axis, and in that said mechanical input device is provided with an actuator which is able to apply a force to the mechanical input device which counters the input from the user.

In one embodiment, the system provides a visible indication of the measured or estimated force applied to the ultrasound transducer. In one embodiment, the force is displayed as a visual indication on the display of the system. In one embodiment, the system is provided with a force feedback mechanism which provides the operator with feedback as to the force which is applied to the ultrasound transducer along the z-axis. The force feedback could be a form of haptic feedback device.

In one embodiment, the input from the user input arrangement representing motion along the Z-axis determines the force along the z-axis which is to be applied to the area to be scanned. In one embodiment, the input from the user input arrangement representing motion along the X and Y-axes determines the velocity of the ultrasound transducer along the X and Y-axes respectively. In one embodiment, the input representing motion along the Z-axis is a mechanical proportional input device which is biased to a centre position with a spring like force against which the user must act to cause the mechanical proportional input device to displace.

In one embodiment, the user input arrangement is provided at a location where the operator is not in direct visual contact with the area to be scanned. In one embodiment, the system can comprise a virtual test environment with a virtual robot and a virtual object to be scanned and the operator can operate the user input arrangement to control the virtual robot in the test environment. In one embodiment, the operator can switch between operating a real robot with a real life object to be scanned and operating a virtual robot with a virtual object to be scanned.

In one embodiment, the user input arrangement allows the user to define an additional orthogonal coordinate system relative to the transducer orthogonal coordinate system and where the desired motion of the transducer can be specified by the user relative to the additional orthogonal coordinate system

In one embodiment, the user input arrangement converts the motion specified by the user in said additional orthogonal coordinate system to motion in the transducer orthogonal coordinate system.

In one embodiment, the motion specified by the user in the additional orthogonal coordinate system is modified by the user input arrangement and/or the controller to maintain the transducer on the 3D model of the surface or at a specified depth with respect to the 3D model of the surface.

It should be noted that while the examples disclosed in this specification all relate to scanning a pregnant woman, the invention according to this specification should not be limited to ultrasound scanning of pregnant women, but can be applied to many different types of applications, for example scanning knee injuries, scanning organs during surgery, scanning animals, etc.

It should be emphasized that the term “comprises/comprising/comprised of” when used in this specification is taken to specify the presence of stated features, integers, steps or components but does not preclude the presence or addition of one or more other features, integers, steps, components or groups thereof.

### BRIEF DESCRIPTION OF THE DRAWINGS

In the following, the invention will be described in greater detail with reference to embodiments shown by the enclosed figures. It should be emphasized that the embodiments shown are used for example purposes only and should not be used to limit the scope of the invention.

FIG. 1 schematically shows an example embodiment of a system according to the current invention.

FIG. 2 shows a flow chart of a first embodiment of a method of controlling the transducer.

FIG. 3 shows a schematic representation of the transducer orthogonal coordinate system defining the motion of the transducer as well as the global fixed coordinate system.

FIG. 4 shows a schematic representation of a system where an additional orthogonal coordinate system is defined relative to a scanning object to allow defining the desired motion of the transducer relative to the scanning object.

### DETAILED DESCRIPTION

### Overview

There are difficulties with prior art systems. For example, in the case where it is desired to scan a pregnant woman, the previously proposed systems are difficult to use since the operation of the transducer is difficult to control for the operator. For example, pre-programed optimized paths are not suitable for an application like this since the operator will need to move the transducer depending on the mother's anatomy as well as the position of the foetus. Here it is not possible to “pre-program” an optimal path.

Using a joystick (the user control device) to control the movements of the robot arm while having to focus on the output of the ultrasound transducer on a display screen (the ultrasound transducer generates an image which is shown to the operator as a live feed on a display) is difficult because the operator cannot follow the location of the transducer and the ultrasound image simultaneously. If a camera is present, then the operator can use the visualization to get visual feedback of where the transducer is located on the area to be scanned. However, correlating this to the motion is difficult. Moving the transducer with a joystick (or any control interface) requires the constant attention on the exact location and orientation of the transducer to then perform the desired movement of the transducer. An example of this is moving from one side of an abdomen of a pregnant woman to the other side without colliding with the abdomen. Other scenarios are scanning the abdomen with a constant angle to the surface.

It should be noted that in this specification the term “operator” is used to describe the person who is operating the transducer and viewing the results of the scan. Furthermore, the term “patient” is used to refer to the person who is being scanned. While the term “patient” typically refers to a person who is sick, in the scope of this specification, the patient does not have to be sick. Likewise, the term “patient” is typically understood as a human being, however, within the scope of the current specification, the “patient” could also be an animal.

### Further Description

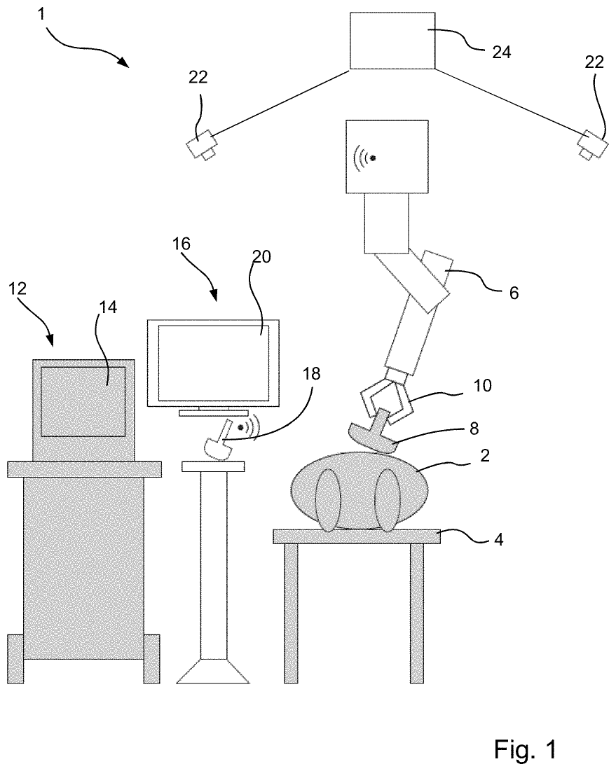

FIG. 1 shows a schematic overview of one embodiment 1 of a system according to the current invention. In the figure, the patient 2 to be scanned is lying on a bench 4. A multi axis robot 6 is arranged above the bench and is holding an ultrasound transducer 8 via a transducer holder 10. The multi axis robot shown in the figures is shown very schematically. The person skilled in the art of robots will know that many different types of multi axis robots are available in the art. For example, in the figure, the robot is shown as a ceiling mounted robot which extends downwardly from the ceiling. However, two other non-limiting examples of possible arrangements are robots mounted on a mobile base or robots mounted on the side of the patient. Similarly, the robot shown is of the articulated type with a number of limbs connected by articulated joints, however other forms of robots are available. Some other forms of robots include Delta robots, SCARA robots, and Cartesian robots. It should be clear that this list is not exclusive.

The system further comprises an ultrasound scanner unit 12 connected to the ultrasound transducer. The scanner unit will typically comprise a display screen 14 showing the output of the ultrasound transducer. A workstation 16 is also provided. The workstation comprises a user input device 18 and a display 20.

In the schematic FIG. 1, the user input device is shown as a simple joystick. However, in a real-world situation, many different types of user input arrangement could be provided. In one case, a user input device having six individual degrees of freedom could be provided. Such types of input device are known in the art, sometimes called 3D Space Balls or 3D space mice. One commercial device is available under the brand 3Dconnexion and is called a “SpaceMouse”. Such devices will have a handle which can be displaced linearly along x, y and z axes as well as rotated about said x, y and z axes. In another embodiment (not shown), the user input arrangement could be provided as two separate joysticks, a first joystick controlling the x, y and z displacements of the transducer and a second joystick controlling the rotations about the x, y and z axes. Other forms of arrangement could also be provided where one joystick defines the motion along the x and y axes while another joystick controls the motion along the z-axis. In one embodiment, the input device controls the motion along the x and y axes by specifying the velocity of the transducer across the surface and the input device controls the motion along the z-axis by specifying the force with which the transducer is pressed against the area to be scanned. In one option, the motion along the z-axis is defined as a velocity until the transducer is in contact with the surface and then via the force once the transducer is in contact with the surface.

The system in the current embodiment further comprises two cameras 22 arranged such that their Field of View (FOV) can capture the area to be scanned of the patient. The system further comprises a vision processing system 24 which combines the images from the cameras to create a 3D image of the scene from which the surface of the area to be scanned, for example the surface of the abdomen of a pregnant woman, is extracted to a 3D model. Other forms of scanners can also be used to get a 3D model of the area to be scanned. One example is a 3D laser scanner. In another option, the robot could be arranged to scan the surface of the area to be scanned with a single axis laser distance measurement (or other form of distance measurement sensor) arranged at the end of the end effector. By moving the robot over the area to be scanned, the 3D model of the surface can be determined. Many options are available to the person skilled in the art to acquire the 3D model of the surface to be scanned.

During the actual ultrasound scanning procedure, the cameras (or other 3D scanning sensors) can be continuously scanning the area to be scanned to identify motion of the patient or any changes in the surface.

The 3D model 100 of the area to be scanned is provided relative to a fixed global orthogonal coordinate system 102 (X₁, Y₁, Z₁). The controller also knows the geometry and position of the multi axis robot in this global coordinate system, such that the controller of the multi axis robot can control the end effector of the multi axis robot relative to the 3D model. Furthermore, the end effector gripper is arranged such that the controller knows the orientation and position of the ultrasound transducer relative to the end effector of the robot. In certain embodiments, different types of ultrasound transducers can be detachably mounted in the gripper and the controller needs to be provided with information on which type of ultrasound transducer is mounted to the gripper, so that the distance between the transducer surface and the end effector of the robot is known.

According to one embodiment of the invention, once the 3D model of the surface is acquired, then the robot will start by moving the ultrasound transducer until the surface of the transducer is placed against the surface of the area to be scanned at a central location. In the case of scanning a pregnant woman, the robot could move the ultrasound scanner to a position centred on the navel of the patient. Since the surface of the area to be scanned is known due to the knowledge of the 3D model, the robot can easily and safely bring the transducer into contact with the surface to be scanned. In some embodiments, the robot can be arranged to stop the transducer a certain distance from the surface, without making direct contact. In other cases, it would be possible for the robot to go into a gravity balanced mode, where the operator can manually move the ultrasound transducer and the robot follows. The operator could then place the transducer in a desired start position manually before starting the remote control.

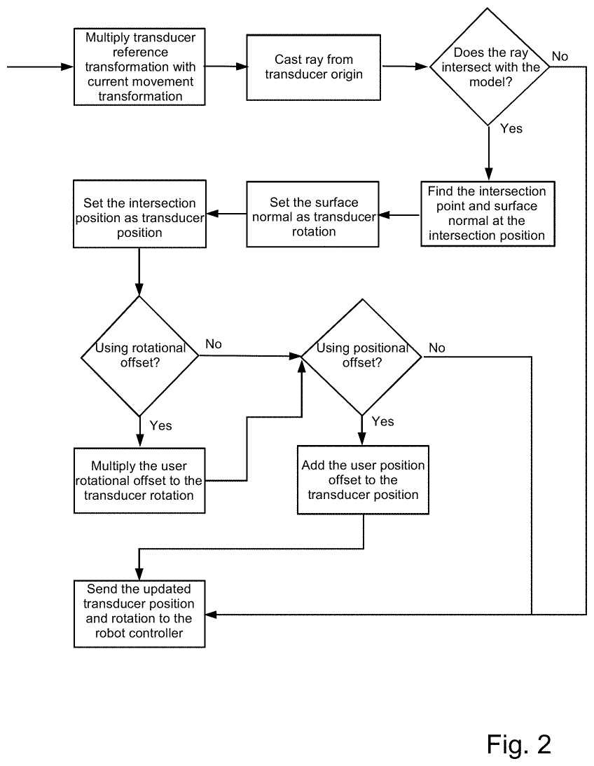

The controller is furthermore arranged to control the orientation and motion of the transducer relative to the 3D model of the surface to be scanned. In a way, the input from the user input device along the X and Y axes follows the 3D model of the surface, while motion along the Z axis will be perpendicular to the surface. In one embodiment, as shown in the flow chart according to FIG. 2, the controller starts by generating a ray which extends out of the end of the transducer until it intersects with the surface of the 3D model. The controller then finds the Normal vector to the surface at the intersection point and rotates the transducer until its central axis is aligned with the normal vector of the surface. The transducer is therefore arranged perpendicular to the surface. Then if the user input device specifies a rotation, the desired rotation is applied to the transducer. Then if the user input device specifies a displacement, the transducer is displaced as indicated by the user input device.

In the current embodiment, the control input device is a joystick with an x and y input motion. By controlling the joystick, the user can easily add a translational offset based on a local (transducer) orthogonal coordinate system 104 (X₂, Y₂, Z₂) defined by the ray and its intersection point with the 3D model. The x and y offsets will be along a plane which is tangent to the surface of the 3D model at the intersection point and the Z input will be perpendicular to the intersection point. Adjusting the x-y inputs of the joystick will move the transducer along the surface. The local coordinate system 104 of the transducer is continuously adjusted according to the 3D model such that the motion will always be along the surface.

Adjusting the z input will move the transducer into or away from the patient. This adjusts the force with which the transducer presses into the skin of the patient. To get a clear ultrasound image, it is often necessary to press the transducer with a significant force against the patient's skin. However, the force applied should not exceed safety limits.

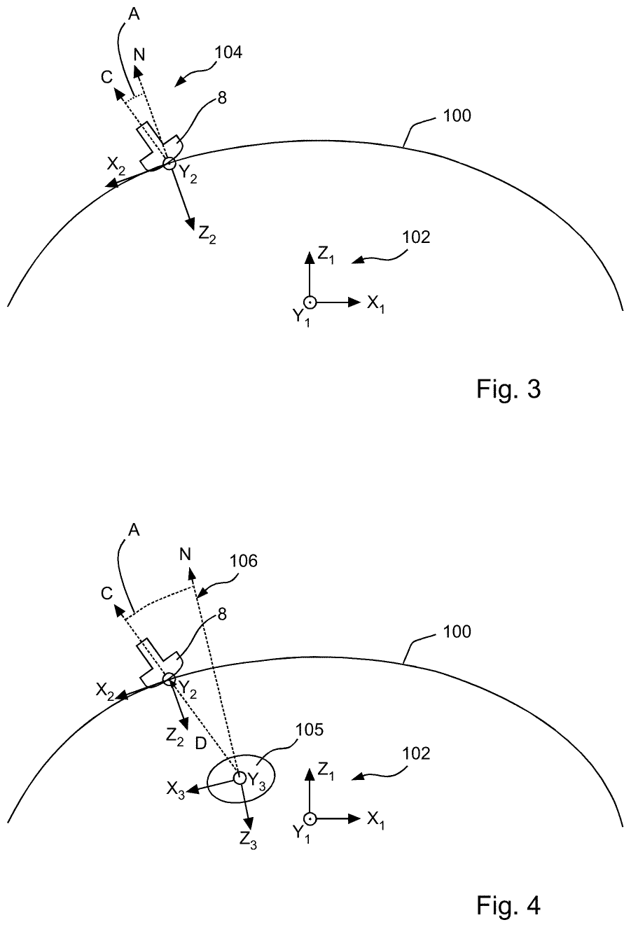

Furthermore, the control input device can also be arranged to provide rotational offsets, which are multiplied with the current rotation of the transducer. As shown in FIG. 3, the transducer 8 has been rotated an angle A about the Y₂ axis such that the central axis C of the transducer is rotated away from the normal vector N of the surface by A degrees. Since the local coordinate system is constantly being updated, then the axes of the local coordinate system will also constantly be being updated. As such the transducer maintains the user provided rotational offset relative to the normal vector of the surface while moving across the 3D surface model.

The resulting behaviour is a system capable of maintaining a reference to the surface positions and the angle of the surface such that the movements of the operator are converted to actual positions of the transducer on the abdomen. A movement from one side of the abdomen to the other can then be performed very easily, just by specifying a translation command from the control interface along one dimension. For example, starting at one side of the abdomen and providing a positive x motion input, the controller will move the transducer along the abdomen while keeping the transducer at the same distance with regards to the surface of the abdomen and at an angle which is always perpendicular to the surface of the abdomen.

In one embodiment, on the display of the workstation, the 3D model is displayed together with an image from the cameras. A virtual model of the transducer is then shown on the screen as well based on the knowledge of the actual position of the transducer. In this way, when the operator is viewing the ultrasound images, the operator can quickly see the visual image of the area to be scanned and the orientation of the transducer without having to entirely move his/her focus away from the ultrasound image. Also the system can show the force being applied to the patient directly on the display via a graphical element, for example a bar graph where the height of the bar illustrates the amount of force applied. In another embodiment, the force applied could be shown by colouring the transducer in different colours depending on the force applied.

In one embodiment, as illustrated in FIG. 4, an additional orthogonal coordinate system (X₃, Y₃, Z₃) is defined relative to an object to be scanned 105. The motion of the transducer can then be specified by the user relative to this additional local coordinate system. However, the controller still maintains the transducer on the surface as described in the procedure above. Hence, the actual motion of the transducer will still be relative to the local coordinate system of the transducer (X₂, Y₂, Z₂), but the operator will be able to control the motion relative to the scanning object.

For example, the rotational angle of the transducer can then be specified relative to the additional local coordinate system. In this way, the operator can easily keep focus on the scanning object without having to position the transducer manually on the surface. For example, in FIG. 4 it can be seen that the transducer is angled at an angle A with respect to the coordinate system of the scanning object. When the user, via the user input device were to reduce the angle A, then instead of just rotating the transducer, the robot would also move the transducer along the surface to keep the focus on the scanning object. The user would not have to control any motion manually, but just change the orientation of the user input device, and the controller would automatically move the transducer as required.

In one embodiment, the system is provided with force sensors on all the actuators/joints of the robot and/or at the end effector. In this way, the force applied to the transducer (or more importantly, the force applied by the transducer to the patient) can be determined. In one embodiment, the force is then reflected to the operator via a feeling of increased resistance to user input device motions. In one concrete embodiment, the user input device comprises a mechanical input device which can be displaced proportionally from a centre position. The more the input device is displaced away from the centre position, the greater the velocity of the transducer. If the transducer experiences resistance against motion, the controller can apply a force to the input device which pushes it towards the centre position. If the operator does not apply an equal counter force, then the input device will move towards the centre and the velocity of the transducer will decrease. In this way, the operator will have a direct feeling of resistance when he or she is controlling the transducer. It should be clear, that the force applied by the input device is lower by a scaling factor than the actual force experienced by the transducer. Otherwise, the operator would experience the same amount of physical stress as in the prior art solution where the operator is manually moving the transducer.

In another embodiment, the force feedback is provided as a form of haptic feedback via a vibration of the control input device. As the force increases, the frequency of the vibration could be increased proportionally. In another embodiment, haptic feedback representative of the force is provided as a tactile surface which stimulates the skin by deforming based on the force measured at the transducer. In another embodiment, the force feedback is provided via a graphical representation of the force on the screen. Vibration could also be used to signal when the transducer reaches the surface of the patient or when the transducer approaches certain points, for example joint limits of the robot, or areas of the patient which need to be avoided.

In another embodiment, since the actual position of the transducer is known and the 3D model of the surface is known, then when the operator presses the transducer into the patient, the system will be able to calculate the distance that the transducer is arranged “below” the surface of the 3D model. This distance can provide an estimate of the force being applied to the person. This could be used as a second source of force estimation in case the force sensors do not provide a sensitive enough reading. Or the estimate could be used in case there are no force sensors which are able to measure the force applied.

It is to be noted that the figures and the above description have shown the example embodiments in a simple and schematic manner. Many of the specific mechanical details have not been shown since the person skilled in the art should be familiar with these details and they would just unnecessarily complicate this description. For example, the specific robot used and the specific control implementations have not been described in detail since it is maintained that the person skilled in the art would be able to find a suitable robot and apply suitable control to implement the system according to the current invention.

### Additional Embodiments

Any of the following Clauses can be implemented:
1. A system for robot-assisted ultrasound scanning comprising:
   a. a multi axis robot with an end effector, said multi axis robot and end effector being arranged such that said end effector can be moved in at least six degrees of freedom,
   b. a transducer holding element for connecting an ultrasound transducer to the end effector of the multi axis robot in a known position with respect to the end effector,
   c. a user input arrangement allowing a user to specify a desired ultrasound transducer displacement along X, Y and Z axes of a transducer orthogonal coordinate system defining the ultrasound transducer motion, and
   d. a controller which is connected to the user input arrangement and which controls the end effector of the multi axis robot based on the input from the user input arrangement,
   characterized
   e. in that the controller acquires a 3D model of a surface to be scanned, and
   f. in that the controller is arranged to continuously update the transducer orthogonal coordinate system defining the ultrasound transducer motion as the ultrasound transducer moves, where the X and Y axes are arranged on a plane which is tangent to a point of intersection between a vector passing through the central axis of the ultrasound transducer and the surface of the 3D model and where the Z axis is arranged along the normal vector to the 3D model at said point of intersection.
2. The system according to Clause 1, characterized in that said user input arrangement comprises at least three separate proportional inputs, said at least three separate proportional inputs representing the desired ultrasound transducer displacement along the X, Y and Z axes of the transducer orthogonal coordinate system defining the ultrasound transducer motion.
3. The system according to Clause 1 or 2, characterized in that the system comprises a 3D scanning arrangement which is arranged to scan a surface of the area to be scanned and to generate a 3D model of the surface of the area to be scanned.
4. The system according to any one of Clauses 1 to 3, characterized in that the user input arrangement has at least one, at least two or at least three additional proportional input(s) representing rotation of the ultrasound transducers about at least one, two or three separate axes respectively and in that the controller is arranged to apply those inputs to rotate the ultrasound transducer about said X, Y and Z axes of the transducer orthogonal coordinate system respectively.
5. The system according to any one of Clauses 1 to 4, characterized in that the system comprises a display, said display displaying a virtual representation of the ultrasound transducer on a representation of the area to be scanned.
6. The system according to Clause 5, characterized in that the system comprises a camera suitable for capturing an image of the area to be scanned and in that the representation of the area to be scanned is the image of the area to be scanned.
7. The system according to any one of Clauses 1 to 6, characterized in that the user input arrangement allows the user to define an additional orthogonal coordinate system relative to the transducer orthogonal coordinate system and in that the desired motion of the transducer can be specified by the user relative to said additional orthogonal coordinate system
8. The system according to Clause 7, characterized in that the user input arrangement converts the motion specified by the user in said additional orthogonal coordinate system to motion in the transducer orthogonal coordinate system.
9. The system according to Clause 7 or 8, characterized in that the motion specified by the user in the additional orthogonal coordinate system is modified by the user input arrangement and/or the controller to maintain the transducer on the 3D model of the surface or at a specified depth with respect to the 3D model of the surface.
10. The system according to any one of Clauses 1 to 9, characterized in that the system further comprises a force sensor which measures the force applied to the ultrasound transducer or a force estimator which estimates the force applied to the ultrasound transducer.
11. The system according to Clause 10, characterized in that the system is arranged to stop motion of the robot when the measured or estimated force exceeds predefined limits and/or in that the system is arranged to reflect the measured or estimated force back to the user via a haptic feedback mechanism in the user input arrangement.
12. The system according to Clause 10 or 11, characterized in that the system provides a visible indication of the measured or estimated force applied to the ultrasound transducer.
13. The system according to any one of Clauses 1 to 12, characterized in that the input from the user input arrangement representing motion along the Z-axis determines the force along the z-axis which is to be applied to the area to be scanned.
14. The system according to any one of Clauses 1 to 13, characterized in that the user input arrangement is provided at a location where the operator is not in direct visual contact with the area to be scanned.

A system for robot-assisted ultrasound scanning comprises a multi axis robot with an end effector, said multi axis robot and end effector being arranged such that said end effector can be moved in at least six degrees of freedom, a transducer holding element suitable for connecting an ultrasound transducer to the end effector of the multi axis robot in a known position with respect to the end effector, a user input arrangement having at least three separate proportional inputs, said at least three separate proportional inputs representing desired ultrasound transducer displacement along X, Y and Z axes of an orthogonal coordinate system defining the ultrasound transducer motion, and a controller which is connected to the user input arrangement and controls the end effector of the multi axis robot, based on the input from the user input arrangement.

### EXAMPLE ALTERNATIVES

In view of the many possible embodiments to which the principles of the disclosed technology can be applied, it should be recognized that the illustrated embodiments are examples of the disclosed technology and should not be taken as a limitation on the scope of the disclosed technology. Rather, the scope of the disclosed technology includes what is covered by the scope and spirit of the following claims.

1. A system for robot-assisted ultrasound scanning comprising:
a. a multi axis robot with an end effector, said multi axis robot and end effector being arranged such that said end effector can be moved in at least six degrees of freedom,
b. a transducer holding element for connecting an ultrasound transducer to the end effector of the multi axis robot in a known position with respect to the end effector,
c. a user input arrangement allowing a user to specify a desired ultrasound transducer displacement along X, Y and Z axes of a transducer orthogonal coordinate system defining the ultrasound transducer motion, and
d. a controller which is connected to the user input arrangement and which controls the end effector of the multi axis robot based on the input from the user input arrangement,
characterized
e. in that the controller acquires a 3D model of a surface to be scanned, and
f. in that the controller is arranged to continuously update the transducer orthogonal coordinate system defining the ultrasound transducer motion as the ultrasound transducer moves, where the X and Y axes are arranged on a plane which is tangent to a point of intersection between a vector passing through the central axis of the ultrasound transducer and the surface of the 3D model and where the Z axis is arranged along the normal vector to the 3D model at said point of intersection.

2. The system according to claim 1, characterized in that said user input arrangement comprises at least three separate proportional inputs, said at least three separate proportional inputs representing the desired ultrasound transducer displacement along the X, Y and Z axes of the transducer orthogonal coordinate system defining the ultrasound transducer motion.

3. The system according to claim 1, characterized in that the system comprises a 3D scanning arrangement which is arranged to scan a surface of the area to be scanned and to generate a 3D model of the surface of the area to be scanned.

4. The system according to claim 1, characterized in that the user input arrangement has at least one, at least two or at least three additional proportional input(s) representing rotation of the ultrasound transducers about at least one, two or three separate axes respectively and in that the controller is arranged to apply those inputs to rotate the ultrasound transducer about said X, Y and Z axes of the transducer orthogonal coordinate system respectively.

5. The system according to claim 1, characterized in that the system comprises a display, said display displaying a virtual representation of the ultrasound transducer on a representation of the area to be scanned.

6. The system according to claim 5, characterized in that the system comprises a camera suitable for capturing an image of the area to be scanned and in that the representation of the area to be scanned is the image of the area to be scanned.

7. The system according to claim 1, characterized in that the user input arrangement allows the user to define an additional orthogonal coordinate system relative to the transducer orthogonal coordinate system and in that the desired motion of the transducer can be specified by the user relative to said additional orthogonal coordinate system

8. The system according to claim 7, characterized in that the user input arrangement converts the motion specified by the user in said additional orthogonal coordinate system to motion in the transducer orthogonal coordinate system.

9. The system according to claim 7, characterized in that the motion specified by the user in the additional orthogonal coordinate system is modified by the user input arrangement and/or the controller to maintain the transducer on the 3D model of the surface or at a specified depth with respect to the 3D model of the surface.

10. The system according to claim 1, characterized in that the system further comprises a force sensor which measures the force applied to the ultrasound transducer or a force estimator which estimates the force applied to the ultrasound transducer.

11. The system according to claim 10, characterized in that the system is arranged to stop motion of the robot when the measured or estimated force exceeds predefined limits and/or in that the system is arranged to reflect the measured or estimated force back to the user via a haptic feedback mechanism in the user input arrangement.

12. The system according to claim 10, characterized in that the system provides a visible indication of the measured or estimated force applied to the ultrasound transducer.

13. The system according to claim 1, characterized in that the input from the user input arrangement representing motion along the Z-axis determines the force along the z-axis which is to be applied to the area to be scanned.

14. The system according to claim 1, characterized in that the user input arrangement is provided at a location where the operator is not in direct visual contact with the area to be scanned.
